(19) United States
(12) Patent Application Publication
(10) Pub. No.: US 2003/0017947 A1
(43) Pub. Date: Jan. 23, 2003

(54) Compositions for protecting plants and method of using the same

(76) Inventor: Hiroshi Kawai, Kanagawa (JP)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/181,248

(22) PCT Filed: Dec. 25, 2000
(86) PCT No.: PCT/JP00/09203

(30) Foreign Application Priority Data
Jan. 20, 2000 (JP) 2000012131

(51) Int. Cl. A61K 31/7016; A01N 43/04; A01N 43/16

(52) U.S. Cl. 504/292; 514/53

(57) ABSTRACT

The plant protective composition of this invention is a composition of at least one selected from the group comprising of trehalose, which is widely found in plants, microorganisms, insects, and fungi, and is thought to have a major role in drought-resistance, cold-resistance in organisms; its derivative, trehalose-6-phosphate, and non-reducing trisaccharide selaginose which has ecological behavior, physical properties close to trehalose; and water-soluble calcium, which is a preventive substance against plant tissue softening, necrosis; which significantly improves plant drought-resistance and cold-resistance by application to plants.

Spraying the plant protective composition of this invention diluted by water up to an upper limit of 10,000 times by volume to plant stem leaves and/or fruit significantly improves drought-resistance, cold-resistance of plants, especially increasing the amount of calcium inside crops, and improving crop yield and merchandise value.

The present invention relates to a plant protective composition and method for its usage, and more specifically a method for its usage aiming to endow drought-resistance and cold-resistance to crops, and prevent physiological disorders of crops due to calcium deficiency, especially providing an effective method to increase the amount of calcium contained in crops and improving the harvest yield and merchandise value.

### BACKGROUND ART

In the farming field, drought harm due to water deficiency and cold damage by low temperature are still a major problem in this era of modern agriculture. Naturally, there is damage to field crops for which temperature and water controls are weather-dependent, but also recently, even damage to crops in indoor cultivation facilities such as greenhouses is increasing because of the effects of changes in cultivated variety, reductions in production costs, insufficient work hands, and the like. Some examples of dryness harm due to insufficient moisture in the soil are withering of the foliage, and scotched leaf margins.

Furthermore, secondary disorders due to dryness of soil are the so called calcium deficiency symptoms such as blossom end rot in tomatoes and green peppers, tipburn in strawberries, core rotting in head vegetables such as Chinese cabbage, fermented fruit in melon, core termination in the growing points, flower bud differentiation disorders, and the like.

In addition, examples of cold harm due to low temperature are yellowing of leaves, root damage, flower bud disorders, abortion of blossoming, fruition disorders, fruit thickening disorders, and the like. It is the present state is that these disorders directly have a significant negative effect on crop yield and quality.

As measures against these harm from drought, low-temperature, a method spraying paraffin and the like to suppress transpiration from leaves (Japanese Patent Laid-Open No.Hei 05-328859), foliar spraying of amino acid fermented solution (Japanese Patent Laid-Open No.Hei 04-58833), spraying of proline, tryptophan (Hokkaido Kamikawa Agricultural Test Fields), and the like have been studied but the reproducibility of results is low, and at times cause drug disorders such as browning and yellowing of foliages, etc.

Additionally, there are plant activators using trehalose (Japanese Patent Laid-Open No.Hei-10-25209), freshness keepers using trehalose and water-soluble chitosan (Japanese Patent Laid-Open No.Hei-11-196765) and the like, but many are limited in their application and/or effect, such as cold-resistance, freshness keeping, and the effect against calcium deficiency due to drought, which especially is a factor that significantly reduces yield and quality of vegetables and grains is still unconfirmed.

In addition, as a measure against calcium deficiency, methods such as foliar spraying of calcium components have been executed from the past, but essentially, the cause of calcium deficiency that occurs in the farm field is almost entirely due to secondary disorders such as insufficient water absorption (fertilizer absorption) due to drought, and the situation is that using these methods that do not place a particular emphasis on endowing drought-resistance to plants do not obtain sufficient effects.

In plants under dry conditions, the initial step is the decrease in the turgor pressure of leave cells and symptoms of withering occur because moisture, which consists about 90% of the leaves, is over-transpirated from stomas. In withered leaves, the translocation of nutrients and water between cells is terminated, and furthermore, consumption of assimilatory nutrients due to respiration increases, resulting in detrimental effects on tissue development. Additionally, as the second step, the calcium supply, which is supposed to be absorbed from the root system by the transpiration flow inside the plant is terminated, leading to softening of tissue and necrosis. This softening of tissue and necrosis is often found where moisture transpiration is essentially low, such as fruit, new leaves, growing points, significantly reducing yield and merchandise value.

On the other hand, under low-temperature conditions, leaves close their pores and suppress the amount of released heat due to transpiration. Suppression of transpiration results in worsening of the translocation of nutrients and water as for dry conditions, inhibiting translocation of nutrients to places where cell division is actively progressing, such as growing points, flowers and the like. In addition, it induces pollen disorders, germ tube disorders, root damage, etc., and results in reduced yield and yield rate.

Conventional measures against the above symptoms are light shields, heat insulators, watering systems, heating systems, etc., but the cost for installing these technologies is a burden for producers, and establishment of an inexpensive, easy-to-do measure especially for crops with low profit rate (profit per 10 are) was a pressing problem.

The first objective of this invention is to solve the problems of the above conventional methods, providing a plant protective composition which endows drought-resistance and cold-resistance to crops and enables prevention of physiological disorders due to calcium deficiency in crops. Furthermore, the second objective is to provide a method of use of this plant protective composition, providing an effective measure to especially increase the calcium content in crops, and improve harvest yield and merchandise value.

### SUMMARY OF THE INVENTION

Taking the background of the field into consideration, as a result of the inventor's intensive studies to solve the problems of the above conventional methods, the inventor has found that application of a composition comprising at least one selected from the group consisting of trehalose, which is widely found in plants, microorganisms, insects, and fungi, and is thought to have a major role in drought-resistance, cold-resistance in organisms; its derivative, trehalose-6-phosphate, and non-reducing trisaccharide selaginose, which has ecological behavior, physical properties close to trehalose; and in addition, water-soluble calcium, which is a preventive substance against plant tissue softening, necrosis, to plants has an effect in improving drought-resistance and cold-resistance in plants, which lead to this invention.

The invention in claim 1 of this invention solving the above problems, relates to a plant protective composition characterized by including water-soluble calcium and at least one selected from the group consisting of trehalose, trehalose-6-phosphate, selaginose as major components.

The invention in claim 2 of this invention relates to the plant protective composition according to claim 1, containing at least one selected from the group consisting of trehalose, trehalose-6-phosphate, and selaginose compound in the range of 0.01 to 30 mass %, and water-soluble calcium compound in the range of 0.1 to 20 mass %, to the whole composition in a stock solution respectively.

The invention in claim 3 of this invention relates to the plant protective composition according to claim 1 or 2, wherein the water-soluble calcium compound is calcium nitrate.

The invention in claim 4 of this invention relates to any of the plant protective compositions according to claims 1 to 3, wherein the water-soluble calcium compound is chelated water-soluble calcium compound.

The invention in claim 5 of this invention relates to any of the plant protective compositions according to claims 1 to 4, which is mixed with one or more adjuvant(s) selected from the group consisting of non-ionic, anionic, cationic, or ampholytic surfactant, fixing agent, thickener, anti-precipitant, neutralizer, preservative, filler, antifoam, and antifreezing agent.

The invention in claim 6 of this invention relates to a method of using plant protective composition according to claim 1 to 5, wherein said plant protective compositions is diluted by water up to an upper limit of 10,000 times by volume and sprayed on plant stem leaves and/or fruit.

The invention in claim 7 of this invention relates to a method of using plant protective composition according to claim 6, wherein said plant protective composition is added with one or more adjuvant(s) selected from the group consisting of non-ionic, anionic, cationic, or ampholytic surfactant, fixing agent, thickener, anti-precipitant, neutralizer, preservative, filler, antifoam, and antifreezing agent, and applied to plants.

The invention in claim 8 of this invention relates to a method of use according to claim 6 or 7, wherein the plant protective composition is used in range of 10 to 2,000 mL per seedling raising box during the seedling raising period in the case of wet-rice cultivation and vegetables.

The invention in claim 9 of this invention relates to a method of use according to any one of claims 6 to 8, wherein the plant protective composition is used in the range of 1 to 500 L per 10 are for field spraying in the case of wet-rice cultivation and vegetables.

The invention in claim 10 of this invention relates to a method of use according to claim 6 or 7, wherein the plant protective composition is applied in 500 mL per seedling raising box every five days from seed sowing during raising of seedling period, and 100 L per 10 are on every 10 days after field transplant in the case of wet-rice cultivation and vegetables.

Below, this invention is described in detail.

Trehalose used in this invention is a type of non-reducing disaccharide comprised of 2 molecules of D-glucose bound together by a 1,1 bond, and there are 3 isomers depending upon the bond form, alpha, alpha-; alpha, beta-; and beta, beta-. The molecular weight is 342.30.

Compared to sucrose and maltose, trehalose has characteristics such as large amounts of nonfreezing water in aqueous solution, low motility of water molecules in the hydration shell, high value of the hydration shell, and high glass-transition point. In living plants, it is used as an emergency energy source, and is an essential substance for tissue framework maintenance, and moreover, meaningful as a nonfreezing solution.

Some examples are that most insects use trehalose as an energy source when taking flight, and it is said that it is thanks to trehalose that dried yeast can restore its living functions by replenishment of water.

Contrary to these basic research studies, cases confirming the effect of using trehalose itself on plants are very rare. The reason is that previously, inexpensive synthesizing methods were not established and that trehalose was a very expensive substance costing a few ten thousand yen per kilogram.

However, recently an enzymatic method using starch allows inexpensive manufacturing, and the situation now is that the use of trehalose is now spreading in many fields. It is possible to synergistically avoid drought disorders, low-temperature disorders by spraying and by affusion with calcium, which has a major role in plant cell softening, necrosis, together with at least one selected from the group consisting of trehalose; its derivative, trehalose-6-phosphate, and selaginose.

Trehalose used in this invention is not limited by the method for production, but one effective embodiment is the method of production by an enzymatic method using E.coli and the like with starch and maltose. Additionally, it is not limited by bond form, but the alpha, alpha- form, which exists in nature, is preferably used.

Trehalose-6-phosphate used in this invention is not limited by the method for production, but one effective embodiment is high-level fermentation of fungus, which is comprising by accumulating large amounts of trehalose, and obtaining trehalose-6-phosphate, a trehalose derivative from the extract. Additionally, it is not limited by bond form, but the alpha, alpha- form, which exists in nature, is preferably used.

Selaginose used in this invention is not limited by the method for production, but one effective embodiment is obtaining selaginose from selaginella plant extracts or anaerobic bacterial extracts confirmed to accumulate large amounts of selaginose. Additionally, it is not limited at all by bond form.

These substances used in this invention may be used separately or as a combination of 2 or more substances.

Water-soluble calcium in this invention can be used without any problems if it is any calcium salt that dissolves in water, such as calcium nitrate, calcium chloride, and the like. Moreover, it is not limited at all by its method of production, form, and the like.

Additionally, water-soluble calcium in this invention may be used by chelating with organic compounds having carboxyl groups, as represented by amino acids such as serine, proline, methionine, tryptophan, and the like, and organic acids represented by citric acid, malic acid, formic acid, and the like. In this instance, it is also not limited at all by the chelating agent.

Singular use and application of inorganic water-soluble calcium to plants may lose its effect by being trapped by organic components in plants such as oxalic acid. On the other hand, use and application of chelated water-soluble calcium to plants prevents being trapped by organic components in plants such as oxalic acid, facilitating translocation of calcium inside the plant body, and gives superior results.

Water-soluble calcium used in this invention may be used separately or as a combination of two or more types.

Against the whole plant protective composition of this invention, the stock solution should preferably contain each of the following: at least one selected from the group consisting of trehalose, trehalose-6-phosphate, and selaginose in the range of 0.01 to 30 mass %, and water-soluble calcium in the range of 0.1 to 20 mass %. It is effective for at least one selected from the group: comprising of trehalose, trehalose-6-phosphate, and selaginose to be in the range of 0.01 to 30 mass %, more preferably from 0.1 to 10 mass %, and especially preferable in the range of 1 to 5 mass %. The effect significantly decreases if it is under 0.01 mass %. Trehalose dissolution rate decreases and handling is more difficult if it is over 30 mass % and so is not preferable.

It is effective for water-soluble calcium to be in the range of 0.1 to 20 mass %, more preferably from 0.2 to 15 mass %, and especially preferable in the range of 1 to 10 mass %. The effect significantly decreases if it is under 0.1 mass t%. Water solubility of water-soluble calcium decreases and productivity worsens and symptoms due to excess occur more readily if it is over 20 mass % and so is not preferable.

Use of calcium nitrate as water-soluble calcium is preferable.

Use of calcium nitrate is especially preferable because of reasons such as: its water solubility is high among water-soluble calcium, the solution is close to neutral pH, ability of simultaneously supplementing nitrogen element, which is one of the 3 primary macronutrients in fertilizers, and does not include chlorine ions, which may transform into substances toxic to plants and the environment. Calcium nitrate is not limited by the method of production, form, and it is possible to use in combination with other calcium salts within the range of keeping the merits mentioned above.

Use of the plant protective composition in this invention is by spraying on the stem leaves and/or fruits of the plant with the plant protective composition in this invention diluted at a upper limit of 10,000 times by volume. The used concentration, spray amount depends on the type of plant, cultivation system, and the like, but dilution of over 10,000 times by volume significantly decreases the effect and is not preferable. Preferably use after dilution in the range of 10 to 5,000 times by volume, more preferably in the range of 100 to 2,000 times by volume.

Use of the plant protective composition in this invention should be by spraying on the stalk leaves and/or fruit of the plant. The method of application in this invention to stem leaves and/or fruit of the plant is not especially limited, examples other than spraying are soaking, applying, and like methods.

Specific embodiments of spraying are, for example, the method of diffusion of the plant protective composition in this invention in water, spraying in the range of 0.5 to 300 liters per 10 are, using power pulverizers, shoulder-hoisted pulverizers, broadcasters, sprayers, manned or unmanned helicopters, aerosolizers, hand sprayers, and the like.

Additionally, spraying, soaking, and applying to stalk leaves and/or fruit, during raising of seedling and after transplant to the growing field all have an effect, but soaking, applying after transfer to growing fields is difficult operationally and also economically disadvantageous, thus, it is preferable to use by spraying the foliage of plants. Preferably, use intervals that are timed with the opening of new leaves, usually 3 to 14 days regularly, however, depending on the stage of growth development, type, growth situations, it is possible to use either everyday or in intervals.

One embodiment of spray amount is as follows. In the case of wet-rice cultivation and vegetables, use plant protective composition of this invention diffused in water in the range of 10 to 2,000 mL per seedling raising box during the seedling raising period. Use below 10 mL is not preferable because the effect significantly decreases. Use of over 2,000 mL is not preferable because effects from excess moisture may occur more readily. Use in the range of 100 to 1,000 mL is preferable, more preferably in the range of 200 to 800 mL.

For field spraying, use in the range of 1 to 500 L per 10 are, preferably in the range of 10 to 300 L, more preferable in the range of 50 to 200 L.

Use of below 1 L is not preferable because the effect significantly decreases. Use of over 500 L may trigger disorders such as leaf scorching, and will take a long time to spray even if pulverizers are used, becoming uneconomical operationally and furthermore, uneconomical because the amount effectively used and applied on plant leaves, etc. is reduced.

The most preferable method for use is to use 500 mL per seedling raising box from every fifth day after seed sowing during raising of seedling, and after transplant to fields, use 100 L per 10 are every 10 days.

The plant protective composition of this invention comprises of singular use or combination of two or more adjuvant(s) selected from the group consisting of non-ionic, anionic, cationic, or ampholytic surfactant, fixing agent, thickener, anti-precipitant, neutralizer, preservative, filler, antifoam, and antifreezing agent, and is further included to apply to plants.

Adjuvant used in this invention is not especially limited, and as long as it does not inhibit the composition for plant stem leaves spraying of this invention, any kind may be used without any problems.

As for surfactants used in this invention, examples of anionic surfactant are: alkyl sulfosuccinate, condensed phosphate, alkyl benzene sulfonate such as dodecyl benzene sodium sulfonate, alkyl naphtalenesulfonate, formalin condensate of naphtalenesulfonate, lignin sulfonate, polycarboxylate, alkyl ether sulfate, polyoxyethylene alkyl aryl phenyl ether sulfate, polyoxyethylene alkyl aryl ether sulfate, polyoxyethylene alkyl aryl sulfate, polyoxyethylene alkyl aryl ether sulfate ester, polyoxyethylene alkyl aryl ether acetate ester, and the like, and examples of its salt are: alkali metal salt, ammonium salt, amine salt, and the like.

Additionally, examples of nonionic surfactants are: polyoxyethylene alkyl ether, polyoxyethylene alkyl aryl ether, polyoxyethylene alkyl aryl phenyl ether, polyoxyethylene styryl phenyl ether, polyoxyethylene alkyl ester, sorbitan alkyl ester, polyoxyethylene sorbitan alkyl ester, polyoxyethylene sorbitan fatty acid ester, polyoxyethylene polyoxypropylene glycol, and the like. In addition, as occasion demands, cationic, ampholytic surfactants may be used.

Examples of fixing agents used in this invention are: D-sorbit, paraffin, casein lime, silicone, starch, resin powder, water swelling high polymer, and the like.

Examples of thickeners used in this invention are: xanthan gum, guar gum, carboxy methyl sodium cellulose acid, colloidal silica, water-soluble polymer compounds such as alpha modified starch, highly purity bentonite, hydrophilic silica, and the like.

Examples of anti-precipitants used in this invention are: sodium alkyl naphthalene sulfonate, formaldehyde condensate of sodium naphthalene sulfonate, anionic humectants and dispersants such as sodium lignin sulfonate, polyoxyalkylene polystyryl phenyl ether, nonionic humectants and dispersants such as polyoxyethylene nonyl phenyl ether, and the like.

Examples of neutralizers used in this invention are: sulfuric acid, hydrochloric acid, sodium hydroxide, potassium hydroxide, and the like.

Examples of preservatives used in this invention are: formalin solution, sodium benzoate, potassium sorbate, para-hydroxybenzoic ester, 1,2- benzothiazoline-3-one, and the like.

Examples of fillers used in this invention are: powder carrier such as clay, talc, calcium carbonate, diatomite, zeolite, bentonite, acid clay, activated clay, attapul gas clay; granular carrier such as vermiculite, perlite, pumice stone; white carbon, potassium chloride, ammonium sulfate, sodium sulfate, powdered cellulose, starch, dextrin, saccharide, rice bran, oil meal, corn feed, wheat bran, and the like.

Additionally, antifoam as represented by silicon, and the like, antifreezing agent, such as ethylene glycol, propylene glycol, and the like, may be added to the plant stem leaves spraying composition in this invention as occasion demands. If the plant stem leaves spraying composition foams, use and application of the proper amount to plants may become difficult. Addition of an effective amount of antifoam to the plant stalk leaves spraying composition of this invention will not only allow use and application of the proper amount to plants, but also facilitate operation by preventing foaming. Addition of an effective amount of antifreezing agent to the plant stalk leaves spraying composition will prevent freezing, having an effect of allowing storage, preservation, use, application during winter and cold regions.

These many be used separately or in any combination of 2 or more types.

### DESCRIPTION OF THE PREFERRED EMBODIMENTS

Below are embodiments to specifically describe this invention, but this invention is not limited by these embodiments.

(EMBODIMENT 1)

TC1 was prepared by putting 560 g of calcium nitrate tetrahydrate (Wako Pure Chemical Industries, Ltd.), 50 g of trehalose (Hayashihara, Co., Ltd.), 100 g of polyoxyethylenesorbitanlaurate into a 1000 mL beaker, and adding water to make 1000 mL.

The density of TC1 is 1.25 and water-soluble calcium concentration was about 7.5 weight %.

Using the same method as for TC1, TC2 was prepared by substituting 50 g trehalose with 30 g trehalose, and further substituting with 20 g trehalose-6-phosphate.

Additionally, TC3 was prepared by substituting trehalose with 98.7% pure selaginose, which was extracted and purified from selaginella.

As an additional comparison, A1 was prepared by dissolving calcium chloride in water, and the water-soluble calcium concentration was adjusted to 7.5 mass %.

(TEST1)

Seeds of cucumber (type: Natsu-suzumi) were sown in 90 mm diameter poly-pots, and 100 plants which have completely open seed leaves per test plot were prepared. The composition of the test plots was set as follows: control plot which is sprayed and treated with tap water, TC1 plot which is treated with TC1, A1 plot which is treated with A1. The watering amount was reduced from the beginning of the test period, and the test was administered with no watering after the tenth day. TC1, A1 treatment solutions were diluted with water 500 times by volume and used in the test. Treatment was done by spraying on the leaves for the first time at the beginning of the test and every 3 days afterwards. 2 weeks after starting treatment, the degree of withering of the stalk leaves in the foliage was evaluated by a 3-step scale, and the average was calculated to evaluate drought-resistance. The degree of withering was 0 for those which do not show any signs of withering, 1 for those with withering of the leaves only, 2 for those with withering of the whole plant. The results of the test are shown in Table 1.

TABLE 1

| Classification | Degree of withering |
|---|---|
| Control plot | 1.65 |
| TC1 plot | 0.55 |
| A1 plot | 1.49 |

As shown clearly from the results in Table 1, significant withering was confirmed at the control plot, A plot which were sprayed with water, but the degree of withering of leaves at the TC1 plot using this invention was low, remaining about one-third. These results are due to the composition comprised of trehalose and water-soluble calcium providing drought-resistant.

(TEST2)

As in Test 1, 100 cucumber seedlings grown in a greenhouse per test plot were prepared, and the composition of the test plots was set as follows: control plot which is sprayed and treated with tap water, TC1 plot which is treated with TC1, A1 plot which is treated with A1. The watering amount was the same as the usual moisture management, and growth was done outside the greenhouse (cultivation sheltered from the rain, maximum temperature 15 degrees Celsius, minimum temperature 7 degrees Celsius) from the tenth day after the beginning of the test. TC1, A1 treatment solutions were diluted by water 500 times by volume and used in the test. Treatment was done by spraying on the leaves for the first time at the beginning of the test and every 3 days afterwards. 2 weeks after starting treatment, the degree of withering of the stem leaves in the above ground portion was evaluated by a 3-step scale, and the average was calculated to evaluate drought-resistance. The degree of withering was 0 for those which do not show any signs of withering, 1 for those with withering of the leaves only, 2 for those with withering of the whole plant. The results of the test are shown in Table 2.

TABLE 2

| Classification | Degree of withering |
|---|---|
| Control plot | 1.40 |
| TC1 plot | 0.78 |
| A1 plot | 1.41 |

As shown clearly from the results in Table 2, significant withering was confirmed at the control plot, A plot which were sprayed with water, but the degree of withering of leaves at the TC1 plot using this invention was low, and there was less yellowing of leaves. These results are due to the composition comprised of trehalose and water-soluble calcium providing drought-resistance.

(TEST 3)

An effect test of this invention on the occurrence of blossom end rot, a representative calcium deficiency symptom in tomatoes was done at a tomato producer's field in Hokota-cho, Ibaraki Pref. The test variety used was “House Momotaro”, and a test plot with 50 plants per plot was set up. From the seedling raising 1.5 leaves period, leaves in each group were sprayed every 7 days with the following: control plot with tap water, TC1 plot with TC1 500 times by volume diluted solution, TC2 plot with TC2 500 times by volume diluted solution, TC3 with TC3 500 times by volume diluted solution, A1 plot with A1 500 times by volume diluted solution, and treatment was also continued after transplant to growing fields. The soil moisture amount was kept slightly low during the whole growth stage, and the percentage of blossom end rot symptoms found in the harvest from the first to fifth set of tomatoes was studied. Results are shown in Table 3.

TABLE 3

| | Blossom end rot |
|---|---|
| Classification | occurrence rate (%) |
| Control plot | 5.8 |
| TC1 plot | 0.7 |
| TC2 plot | 0.5 |
| TC3 plot | 0.8 |
| A1 plot | 4.9 |

As shown clearly from the results in Table 3, spraying of this invention reduces blossom end rot (calcium deficiency syndrome) of tomatoes which commonly occurs under relatively dry conditions. Spraying of A1, which was done for comparison, showed almost no effect, clearly showing that singularly sprayed water-soluble calcium has almost no effect.

(TEST 4)

An effect test of this invention on the effect against core termination in komatsuna (Brassica campestris.var. Komatsuna) was done at a field in Tajiri-cho, Onda-gun, Miyagi Pref. The test plot was set to 30 square meters per plot. From the opening of true leaves, leaves in each group were sprayed every 5 days with the following: control plot with tap water, TC1 plot with TC1 500 times by volume diluted solution, TC2 plot with TC2 500 times by volume diluted solution, TC3 with TC3 500 times by volume diluted solution, A1 plot with A1 500 times by volume diluted solution; and the percentage of core termination symptoms found at harvest was studied. Results are shown in Table 4.

TABLE 4

| | Core termination |
|---|---|
| Classification | occurrence rate (%) |
| Control plot | 5.5 |
| TC1 plot | 1.3 |
| TC2 plot | 0.8 |
| TC3 plot | 0.8 |
| A1 plot | 5.2 |

As shown clearly from the results in Table 4, spraying of this invention reduces komatsuna core termination which commonly occurs under conditions of low moisture and insufficient sunlight. Spraying of A1, which was done for comparison, showed almost no effect, clearly showing that singularly sprayed water-soluble calcium has almost no effect.

Effects of the Invention

The plant protective composition of this invention is a composition of at least one selected from the group comprising of trehalose, which is widely found in plants, microorganisms, insects, and fungi, and is thought to have a major role in drought-resistance, cold-resistance in organisms; its derivative, trehalose-6-phosphate, and non-reducing trisaccharide selaginose which has ecological behavior, physical properties close to trehalose; and water-soluble calcium, which is a preventive substance against plant tissue softening, necrosis; which significantly improves plant drought-resistance and cold-resistance by application to plants.

Spraying the plant protective composition of this invention diluted by water up to an upper limit of 10,000 times by volume to plant stem leaves and/or fruit, significantly improves drought-resistance, cold-resistance of plants, especially increasing the amount of calcium inside crops, and improving crop yield and merchandise value.

Industrial Applicability

Use of the plant protective composition of this invention on plants significantly improves drought-resistance, cold-resistance. Spraying the plant protective composition of this invention diluted by water up to an upper limit of 10,000 times by volume to plant stem leaves and/or fruit, significantly improves drought-resistance, cold-resistance of plants, especially increasing the amount of calcium inside crops, and improving crop yield and merchandise value, thus useful for industrial purposes.

What is claimed is:

1. A plant protective composition containing as major ingredients: water-soluble calcium compound and at least one selected from the group consisting of trehalose, trehalose-6-phosphate, and selaginose.

2. A plant protective composition according to claim 1, containing at least one selected from the group consisting of trehalose, trehalose-6-phosphate, and selaginose compound in the range of 0.01 to 30 mass %, and water-soluble calcium compound in the range of 0.1 to 20 mass %, to the whole composition in a stock solution respectively.

3. A plant protective composition according to claim 1 or 2, wherein the water-soluble calcium compound is calcium nitrate.

4. A plant protective composition according to any of claims 1 to 3, wherein the water-soluble calcium compound is chelated water-soluble calcium compound.

5. A plant protective composition according to any of claims 1 to 4, which is mixed with one or more adjuvant selected from the group consisting of non-ionic, anionic, cationic, or ampholytic surfactant, fixing agent, thickener, anti-precipitant, neutralizer, preservative, filler, antifoam, and antifreezing agent.

6. A method of using plant protective composition according to claim 1 to 5, wherein said plant protective composition is diluted by water up to an upper limit of 10,000 times by volume and sprayed on plant stem leaves and/or fruit.

7. A method of using plant protective composition according to claim 6, wherein said plant protective composition is added with one or more adjuvant selected from the group consisting of non-ionic, anionic, cationic, or ampholytic surfactant, fixing agent, thickener, anti-precipitant, neutralizer, preservative, filler, antifoam, and antifreezing agent and applied to plants.

8. A method of use according to claim 6 or claim 7, wherein the plant protective composition is used in the range of 10 to 2,000 mL per seedling raising box during seedling raising period in the case of wet-rice cultivation and vegetables.

9. A method of use according to any one of claims 6 to 8, wherein the plant protective composition is used in the range of 1 to 500 L per 10 are for field spraying in the case of wet-rice cultivation and vegetables.

10. A method of use according to claim 6 or 7, wherein the plant protective composition is applied in 500 mL per seedling raising box every 5 days from seed sowing during raising of seedling period, and 100 L per 10 are on every ten days after field transplant in the case of wet-rice cultivation and vegetables.
